(19) United States
(12) Patent Application Publication
(10) Pub. No.: US 2022/0054421 A1
(43) Pub. Date: Feb. 24, 2022

(54) Process for Preparing Polymeric Particles

(71) Applicant: Evonik Operations GmbH, Essen (DE)

(72) Inventors: Manfred AßMUS, Bickenbach (DE); Thomas Endres, Iphofen (DE)

(73) Assignee: Evonik Operations GmbH, Essen (DE)

(21) Appl. No.: 17/309,498

(22) PCT Filed: Nov. 11, 2019
(86) PCT No.: PCT/EP2019/080788

§ 371 Date: Jun. 2, 2021

(30) Foreign Application Priority Data
Dec. 5, 2018 (EP) 18210330.9

(51) Int. Cl. A61K 9/50; C08F 220/14; C08J 3/12

(52) U.S. Cl. CPC A61K 9/5026; C08F 220/14; C08J 2333/10; C08F 2800/20; C08J 3/126

(57) ABSTRACT

A process for preparing polymeric particles involves a stepwise or gradient emulsion polymerization. The polymeric particles contain polymerized units of methacrylic acid and further monomers, with an overall monomer composition by weight of polymerized units of 5 to 25% by weight of methacrylic acid and 75 to 95% by weight of further monomers. The further monomers are selected from C₁- to C₄-alkylesters of methacrylic acid and/or C₁- to C₄-alkylesters of acrylic acid. The ratio by weight of polymerized units of methacrylic acid to further monomers increases stepwise or in a gradient from the center to the surface of the particles and the polymeric particles are obtained in the form of an aqueous dispersion.

### FIELD OF THE INVENTION

The invention relates to a process for preparing polymeric particles by stepwise or gradient emulsion polymerization.

### BACKGROUND

U.S. Pat. No. 5,644,011 describes a coating and binder composition for pharmaceutical agents. The coating or binder is a (meth)acrylate copolymer produced by emulsion polymerization in the form of an aqueous dispersion and may have a composition of (A) about 10-25 wt-% methacrylic acid, (B) about 40-70 wt.-% methyl acrylate, and (C) 20-40 wt-% methyl methacrylate, based on a total copolymer weight of 100 wt-%. A copolymer polymerized from 10% by weight methacrylic acid, 65% by weight methyl acrylate, and 25% by weight methyl methacrylate is mentioned in U.S. Pat. No. 5,644,011 example B2.

WO 2012/171575A1 describes a coating composition suitable for the coating of a pharmaceutical or nutraceutical dosage form, comprising a core comprising one or more pharmaceutical or nutraceutical active ingredients, wherein the coating composition is comprising at least 20% by weight of an enteric core/shell polymer composition derived from an emulsion polymerization process, wherein either the core of the core/shell polymer composition is formed by a water-insoluble, not cross-linked polymer or copolymer and the shell of the core/shell polymer composition is formed by an anionic polymer or copolymer or vice versa.

Suitable anionic (meth)acrylate copolymers may be those composed of 40 to 60% by weight methacrylic acid and 60 to 40% by weight methyl methacrylate or 60 to 40% by weight ethyl acrylate (EUDRAGIT® L or EUDRAGIT® L100-55 types). EUDRAGIT® L is a copolymer of 50% by weight methyl methacrylate and 50% by weight methacrylic acid. The pH of the start of the specific active ingredient release in intestinal juice or simulated intestinal fluid is about pH 6.0. EUDRAGIT® L 100-55 is a copolymer of 50% by weight ethyl acrylate and 50% by weight methacrylic acid. EUDRAGIT® L30 D-55 is a dispersion comprising 30% by weight EUDRAGIT® L 100-55. The pH of the start of the specific active ingredient release in intestinal juice or simulated intestinal fluid is about pH 5.5.

Likewise suitable are anionic (meth)acrylate copolymers composed of 20 to 40% by weight methacrylic acid and 80 to 60% by weight methyl methacrylate (EUDRAGIT® S type). The pH of the start of the specific active ingredient release in intestinal juice or simulated intestinal fluid is about pH 7.0.

Suitable (meth)acrylate copolymers are those consisting of 10 to 30% by weight methyl methacrylate, 50 to 70% by weight methyl acrylate and 5 to 15% by weight methacrylic acid (EUDRAGIT® FS type). The pH at the start of the specific active ingredient release in intestinal juice or simulated intestinal fluid is about 7.0. EUDRAGIT® FS is a copolymer of 25% by weight methyl meth-acrylate, 65% by weight methyl acrylate and 10% by weight methacrylic acid. EUDRAGIT® FS 30 D is a dispersion comprising 30% by weight EUDRAGIT® FS type copolymer.

In some cases the release behavior of the coating compositions employing the core/shell polymer compositions as described in WO 2012/171575A1 may differ from that of the corresponding non-inventive enteric coatings. For instance in some cases it was observed that when the EUDRAGIT® FS type polymer is used in a certain core/shell polymer composition as disclosed in WO 2012/171575 A1, the release of the active ingredient starts already at pH 6.8 and is faster while the start of the release with the corresponding polymer mixture is around pH 7.0 is slower. It has to be noted however, that a reduction of the active ingredient release at pH 6.8 is estimated to be insufficient for the purposes to the present invention.

EUDRAGIT® L 100 and EUDRAGIT® L 100-55 are well-known commercially available (meth)acrylate copolymer products for pharmaceutical applications.

EUDRAGIT® L 100 is a copolymer polymerized from 50% by weight methyl methacrylate and 50% by weight methacrylic acid. The pH of the start of the specific active ingredient release in intestinal juice or simulated intestinal fluid can be stated to be at about pH 6.0.

EUDRAGIT® L 100-55 is a copolymer polymerized from 50% by weight ethyl acrylate and 50% by weight methacrylic acid. EUDRAGIT® L 30 D-55 is a dispersion comprising 30% by weight EUDRAGIT® L 100-55. The pH of the start of the specific active ingredient release in intestinal juice or simulated intestinal fluid can be stated to be at about pH 5.5.

Likewise suitable are anionic (meth)acrylate copolymers polymerized from 20 to 40% by weight methacrylic acid and 80 to 60% by weight methyl methacrylate (EUDRAGIT® S type). The pH of the start of the specific active ingredient release in intestinal juice or simulated intestinal fluid can be stated to be at about pH 7.0.

EUDRAGIT® FS 30 D is a well-known commercially available (meth)acrylate copolymer product for pharmaceutical applications in the form of a 30% by weight aqueous dispersion. The copolymer is polymerized from 10% by weight methacrylic acid, 65% by weight methyl acrylate, and 25% by weight methyl methacrylate and thus corresponds to U.S. Pat. No. 5,644,011 example B2. The molecular weight is about 280,000 g/mol.

### SUMMARY OF THE INVENTION

EUDRAGIT® L 100 and EUDRAGIT® L 100-55 are well-known commercially available (meth)acrylate copolymer products for pharmaceutical applications. EUDRAGIT® L 100 is a copolymer polymerized from 50% by weight methyl methacrylate and 50% by weight methacrylic acid. The pH of the start of the specific active ingredient release in intestinal juice or simulated intestinal fluid is about pH 6.0. EUDRAGIT® L 100-55 is a copolymer polymerized from 50% by weight ethyl acrylate and 50% by weight methacrylic acid. The pH of the start of the specific active ingredient release in intestinal juice or simulated intestinal fluid is about pH 5.5.

Nutraceuticals like vitamins are usually intended to be released right after the stomach in the small intestine. Due to the start of the specific active ingredient release in intestinal juice or simulated intestinal fluid at about pH 5.5 respectively at about pH 6.0, EUDRAGIT® L 100 or EUDRAGIT® L 100-55 would be suitable as a coating or binding material for nutraceutical applications as well. However since nutraceuticals are sold freely without the control of a prescription like pharmaceuticals, the daily intake of these polymers with comparatively high methacrylic acid content cannot be controlled in a proper way. Individuals may take higher daily dosages than recommended by the manufacturer and thus might overdose the polymers with high methacrylic acid content, which should be avoided to exclude undesired side effects. The invention is also applicable for pharmaceuticals where there is a general trend to reduce the total amount of carboxylic groups in a coating formulation or in a polymeric matrix formation but the active ingredient release is intended to start already in the range of pH 5.8 to 6.5.

EUDRAGIT® FS is a copolymer polymerized from 10% by weight methacrylic acid, 65% by weight methyl acrylate, and 25% by weight methyl methacrylate which would make it suitable for nutraceuticals as the content of methacrylic acid groups is five times lower than that in EUDRAGIT® L 100 or EUDRAGIT® L 100-55. However the pH at the start of the specific active ingredient release of the EUDRAGIT® FS polymer in intestinal juice or simulated intestinal fluid is around pH 7.0 which is too high for the intended release of nutraceuticals which is about 5.8 to 6.3.

Thus there is a need for a polymer for nutraceutical applications with a the specific active ingredient release in intestinal juice or simulated intestinal fluid already around pH 6 but with overall comparatively low amount of methacrylic acid groups in the polymer.

Disclosed is a process for preparing polymeric particles, comprising polymerized units of methacrylic acid and further monomers, with an overall monomer composition by weight comprising polymerized units of 5 to 25% by weight methacrylic acid and 75 to 95% by weight of further monomers, wherein the further monomers are selected from C1- to C4-alkylesters of methacrylic acid and/or C1- to C4-alkylesters of acrylic acid, by stepwise or gradient emulsion polymerization, wherein the ratio by weight of polymerized units of methacrylic acid to further monomers is increasing stepwise or in a gradient from the center to the surface of the particles and wherein the polymeric particles are obtained in the form of an aqueous dispersion.

The term “from the center to the surface of the particles” shall mean, assuming a round respectively a spherical particle, a direct way from the midpoint inside the polymeric particle (center) to (towards) the outside (surface) of the particle. The content of polymerized units of methacrylic acid increases from the center to the surface of the polymeric particle.

The polymer particles resulting from the disclosed process are deemed by the inventors to show an increased concentration of the carboxylic groups of the polymerized units of methacrylic acid on their surface compared to their allover methacrylic acid content. Although the allover methacrylic acid content is comparatively low, it seems that the polymer particles as disclosed, when used as a coating or binding material in dosage forms comprising an active ingredient, act like copolymers or copolymer particles with much higher content of methacrylic acid. Thus, a process for preparing polymer particles with comparatively low allover methacrylic acid content and an unexpected low dissolution and active ingredient release behavior at the same time is provided. The invention also discloses the polymer particles and their use as coating or binding agent in a pharmaceutical or nutraceutical dosage form.

### DETAILED DESCRIPTION OF THE INVENTION

### Process

Disclosed is a process for preparing polymeric particles, comprising polymerized units of methacrylic acid and further monomers, with an overall monomer composition by weight comprising polymerized units of 5 to 25% by weight methacrylic acid and 75 to 95% by weight of further monomers, wherein the further monomers are selected from C1- to C4-alkylesters of methacrylic acid and/or C1- to C4-alkylesters of acrylic acid, by stepwise or gradient emulsion polymerization, wherein the ratio by weight of polymerized units of methacrylic acid to further monomers is increasing stepwise or in a gradient from the center to the surface of the particles and wherein the polymeric particles are obtained in the form of an aqueous dispersion.

Polymeric particles with the same overall monomer composition by weight may be polymerized altogether simultaneously (not according to the invention=batch emulsion or standard one step polymerization process) or stepwise or in a gradient (according to the invention). The overall monomer composition by weight is constant for a certain polymer or polymeric particle at the end of the stepwise or the gradient emulsion processes as described herein.

In contrast to the overall monomer composition by weight, which is always constant for a certain polymer or polymeric particle, the ratio by weight of methacrylic acid to the further monomers is not constant within the particles from the center to the surface and also not constant at any time during the stepwise or gradient emulsion processes as described herein. At the end of these processes however the overall monomer composition by weight of the monomers in respect to the polymeric particle as a whole is achieved.

The difference of the process as disclosed to a “batch or standard one step emulsion polymerization process” is however that the ratio by weight of polymerized units of methacrylic acid to further monomers is increasing stepwise or in a gradient from the inside towards the outside of the particles. From the inside to the outside of the particles shall mean along the way or the distance from the center towards respectively to the surface of the particles.

The process as disclosed may be characterized in that the polymeric particles are comprising an overall monomer composition by weight comprising polymerized units of 10 to 30% by weight methyl methacrylate, 50 to 70% by weight methyl acrylate and 5 to 15% by weight methacrylic acid as an overall percentage by weight. The ratio by weight of polymerized units of methacrylic acid to the further monomers methyl methacrylate and methyl acrylate is thereby increasing stepwise or in a gradient from the inside (center) of the particles to the outside (surface) of the particles.

According to the disclosed process, the monomers become uneven distributed within the polymeric particles. The distribution of the polymerized units of methacrylic acid is increasing stepwise or in a gradient from the inside to the outside of the particles. Thus the concentration of polymerized units of methacrylic acid on the outside or the surface of the polymeric particles is higher than inside. This uneven distribution of the polymerized units of methacrylic acid is apparently important for the modified function of the polymeric particles, compared to “conventional” polymeric particles from a batch emulsion process with the same monomer composition but with even or nearly even distribution of the polymerized monomers within the polymeric particle. The even or nearly even distribution of the monomers in “conventional” polymeric particles is achieved when the monomers are polymerized altogether in one step. The overall monomer composition by weight may however be the same in inventive and non-inventive polymeric particles.

The uneven distribution of the monomers within the particles may be achieved by stepwise or gradient emulsion polymerization.

### Emulsion Polymerization Process

An emulsion polymerization process may advantageously be carried out by the monomer emulsion feed process or the monomer feed process, respectively in a polymerization reactor. For this, water is heated to the reaction temperature in the polymerization reactor. Surfactants and/or initiators may be added at this stage. Then, depending on the mode of operation, a monomer or a monomer mixture or an emulsion of either are fed to the reactor. This dosed liquid may contain initiators and/or surfactants or the initiator and/or the surfactant may be dosed in parallel.

Alternatively, all monomers can be charged into the reactor, before adding the initiator. This method is often referred to as “batch emulsion process” (not according to the invention).

It is also possible to do a combination of both processes, by polymerizing a part of the monomers in the manner of a batch process, and feeding the other part afterwards. As known to the expert in the field, the type of process and mode of operation can be chosen to achieve the desired particle size, sufficient dispersion stability, a stable production process and so on.

Emulsifiers which may be used are especially anionic and non-ionic surfactants. The amount of emulsifier used is generally not more than 5% by weight, preferably 0.1 to 3% by weight based on the weight of the monomers. Typical emulsifiers are for example alkyl sulfates (e.g. sodium dodecyl sulfate), alkyl ether sulfates, dioctyl sodium sulfosuccinate, polysorbates (e.g. polyoxyethylene (20) sorbitan monooleate), nonylphenol ethoxylates (nonoxynol-9) and others.

Besides those polymerization initiators conventionally used in emulsion polymerization (e.g. per-compounds, such as ammonium peroxodisulfate, (APS) redox systems, such as sodium disulphite-APS-iron can be applied. Also water soluble azo initiators may be applied and/or a mixture of initiators can be used. The amount of initiator is usually between 0.005 to 0.5, 0.01 to 0.3% by weight, based on the weight of the monomers.

A chain transfer agent may be added to improve the process stability and the reproducibility of the molecular weight (Mw). A typical amount of chain transfer agent may be 0.05 to 1% by weight based on monomer weight. A typical chain transfer agent may be, for example, thioglycolic acid 2-ethyl hexyl ester (TGEH) or n-dodecyl mercaptan (nDDM). However, the chain transfer agent may be omitted in many cases, without affecting the properties according to the invention.

A typical emulsion polymerization broth may comprise the monomers and water at a typical ratio by weight of about 3 to 7 as main components and 0.005 to 0.5% by weight of one more polymerization initiator(s), 0.05 to 1% by weight one more chain transfer agent(s), less than 5% by weight or 0.1 to 3.0% by weight of one or more emulsifier(s) and 0 to 0.5% by weight of an antifoam agent, wherein all components may add up to 100%.

In a typical core/shell emulsion polymerization process, first a core in the form of a core particle is formed by polymerization of the monomers required for the polymer or copolymer of the core. Subsequently the monomers for the polymer or copolymer of the shell are polymerized in the same reaction mixture to give a shell around respectively on the surface of the core particles.

It may be as well possible to start the emulsion polymerization process first by the addition of readily polymerized polymer particles, such as cellulose particles or starch particles, to the polymerization mixture. Subsequently, the monomers required for polymer or the copolymer of the shell are polymerized in this reaction mixture to give the shell around on the surface of the readily polymerized polymer core particles.

The polymerization temperature depends on the initiators within certain limits. For example, if APS is used, it is advantageous to operate in the range from 60 to 90° C.; if redox systems are used it is also possible to polymerize at lower temperatures, for example at 30° C.

At the end of the process the reactor content is usually allowed to cool down, for instance to 20 to 25° C. and the resulting dispersion may be filtered, for instance through a 250 μm gaze.

The average particle size (D50) of the polymeric particles produced in the emulsion polymerization may range from 50 to 500 or 80 to 300 nm. The average particle size of the polymer particles may be determined by methods well known to a skilled person for instance by the method of laser diffraction. The particle size may be determined by laser diffraction, using a Mastersizer 2000 (Malvern). The values can be indicated as particle radius rMS [nm], which is half of the median of the volume based particle size distribution d(v,50).

The obtained dispersion can directly be used to prepare a coating suspension, or, in rare cases, be used as coating suspension without even adding further excipients.

The dispersion can also be dried to a powder or granulate, preferably by spray drying, spray granulation, freeze drying, coagulation or extrusion. Thus, a solid powder or granulate can be obtained, which offers certain advantages with regard to handling and logistics. The dry powder or granulate may be used as polymeric binder for matrix dosage forms.

The dried polymerizate may then be transferred into a coating suspension by redispersing the solid in water, e.g. (where required) by the use of a high shear mixer.

### Stepwise Emulsion Polymerization

When the process is a stepwise emulsion polymerization, the process may comprise at least a first and a second step, wherein in the first step polymeric core particles are polymerized, wherein the ratio by weight of methacrylic acid to the further monomers is lower compared to the overall monomer composition by weight of methacrylic acid to the further monomers and wherein in a second step a polymeric shell is polymerized onto the polymeric core wherein the ratio by weight of methacrylic acid to the further monomers is higher compared to the overall monomer composition by weight of methacrylic acid to further monomers.

Although a two-step process is preferred, it is evident that the stepwise polymerization process may be carried out also in more than two steps, wherein in the last step the polymeric shell is polymerized onto the polymeric core generated in the previous steps, wherein the ratio by weight of methacrylic acid to the further monomers is higher compared to the overall monomer composition by weight of methacrylic acid to further monomers.

In one embodiment of the invention the process may be a stepwise emulsion polymerization with two steps, wherein in the first step the further monomers, preferably methyl methacrylate and methyl acrylate, are polymerized as polymeric core particles and wherein in the second step the methacrylic acid is added and polymerized as polymeric shell onto the polymeric core particles.

At the end of the process the reactor content is usually allowed to cool down, for instance to 20 to 25° C., and the resulting dispersion may be filtered, for instance through a 250 μm gaze.

### Gradient Emulsion Polymerization

When the process is a gradient emulsion polymerization, the monomers are polymerized in a continuous process, wherein the ratio by weight of the methacrylic acid to the further monomers is continuously increased during the polymerization process. The term “during the polymerization process” shall mean the time interval from the beginning of the process, the polymerization initiation, until the end of the process, when a polymerization degree of 95% by weight or more, preferably of 98% by weight or more of monomer to polymer conversion has been achieved.

The monomers may be polymerized in a continuous process, starting with the polymerization of an initial excess of the further monomers to methacrylic acid in relation to the intended overall monomer ratio by weight of the monomers. Thus, at the beginning of the process, the further monomers, preferably methyl methacrylate and methyl acrylate, are polymerized under addition of an initial shortfall of the methacrylic acid or even without any addition of methacrylic acid. During the further polymerization process until its end, the residual amount of methacrylic acid is added constantly respectively increasingly until totally consumed. As an example, the polymerization process is initiated in the presence of the total amount of the further monomers only while methacrylic acid is added constantly over the remaining time, e.g. dropwise, to the polymerization broth until a polymerization degree of 95% by weight or more, preferably of 98% by weight or more of monomer to polymer conversion may be achieved.

At the end of the process the reactor content is usually allowed to cool down, for instance to 20 to 25° C., and the resulting dispersion may be filtered, for instance through a 250 μm gaze.

### General Example for a Gradient Emulsion Polymerization

A general example for a gradient emulsion polymerization may be as follows:

22 to 28% by weight methyl methacrylate and
62 to 68% by weight methyl acrylate are mixed and continuously charged into water.

During the charge 7 to 13% by weight methacrylic acid are continuously charged into the methyl methacrylate and methyl acrylate mixture. The monomers, which add up to 100%, polymerize and form a 20 to 40% by weight aqueous dispersion.

As excipients for the polymerization sodium persulfate, 2-ethylhexylthioglycolate, sodium dodecyl sulfate and Polysorbate 80 may be used.

This general process results in an aqueous dispersion comprising polymeric particles with a continuously varying monomer composition from the center to the surface of the particles. The continuously increasing addition of methacrylic acid to methyl methacrylate and methyl acrylate can be calculated from the beginning to the end of the process. The methacrylic acid content rises from 0% or nearly 0% in the center of the polymeric particles to approximately 38 to 42% by weight at or near to the surface of the polymeric particles. The overall monomer composition of the polymeric particles is however equal to polymerized 7 to 13% by weight methacrylic acid, 22 to 28% by weight methyl methacrylate and 62 to 68% by weight methyl acrylate, wherein the monomers add up to 100%.

Specific Example for a Gradient Emulsion Polymerization

A specific example for a gradient emulsion polymerization may be as follows:

25% by weight (7.46 g) methyl methacrylate and
65% by weight (19.29 g) methyl acrylate are mixed and continuously charged into 69.8 g of water.

During the charge 10% by weight (2.82 g) methacrylic acid are continuously charged into the methyl methacrylate and methyl acrylate mixture. The monomers add up to 100% and polymerize and form a 30% by weight aqueous dispersion.

As excipients 0.07 g sodium persulfate, 0.08 g 2-ethylhexylthioglykolate, 0.1 g sodium dodecyl sulfate and 0.35 g Polysorbate 80 are used.

This specific process results in an aqueous dispersion comprising polymeric particles with a continuously varying monomer composition from the center to the surface of the particles. The continuously increasing addition of methacrylic acid to methyl methacrylate and methyl acrylate can be calculated from the beginning to the end of the process. The methacrylic acid content rises from about 0% in the center of the polymeric particles to approximately 40% by weight at or near to the surface of the polymeric particles. The overall monomer composition of the polymeric particles is however equal to polymerized 10% by weight methacrylic acid, 25% by weight methyl methacrylate and 65% by weight methyl acrylate, wherein the monomers add up to 100%.

### Polymeric Particle

Disclosed is a polymeric particle, obtainable in the process as described herein, comprising a stepwise or continuous increase of polymerized methacrylic acid units from the center to the surface. From the inside to the outside of the particles shall mean along the way or the distance from the center to the surface of the particles.

The polymeric particle is comprising polymerized units of 5 to 25% by weight of methacrylic acid and 75 to 95% by weight of further monomers, wherein the further monomers are selected from C1- to C4-alkylesters of methacrylic acid and C1- to C4-alkylesters of acrylic acid. Methacrylic acid and further monomers add up to 100%. The preferred further monomers are methyl methacrylate and methyl acrylate.

The Polymeric particle is preferably comprising polymerized units of 10 to 30% by weight methyl methacrylate, 50 to 70% by weight methyl acrylate and 5 to 15% by weight methacrylic acid. Methyl methacrylate, methyl acrylate and methacylic acid may add up to 100%.

The polymeric particle may have an average particle size (d50) in the range from about 50 to 500, preferably from about 80 to 300 nm.

The determination of the average particle size (d50) may be performed by laser diffraction according to the United States Pharmacopeia 36 (USP) chapter <429> and European Pharmacopeia 7.0 (EP) chapter 2.9.31. The laser diffraction method is based on the phenomenon that particles scatter light in all directions with an intensity pattern that is dependent on particle size. A representative sample, dispersed at an adequate concentration in a suitable liquid or gas, is passed through the beam of a monochromic light source, usually from a laser. The light, scattered by the particles at various angles, is measured by a multi-element detector, and numerical values relating to the scattering pattern are then recorded for subsequent analysis. The numerical scattering values are then transformed, using an appropriate optical model and mathematical procedure, to yield the proportion of total volume to a discrete number of size classes forming a volumetric particle size distribution (e.g. d50 describes a particle diameter corresponding to 50% of cumulative undersize distribution).

The polymeric particle as disclosed may be characterized in that, the increasing concentration of polymerized units of methacrylic acid from the center to the surface of the particle results in an accelerated dissolution speed compared to a polymeric particle, polymerized by an emulsion polymerization in one step.

The polymeric particle as disclosed may be characterized in that, the increasing concentration of polymerized units of methacrylic acid from the center to the surface of the particles results in a lowered active ingredient release pH of an active ingredient containing coated composition or an active ingredient containing polymeric matrix composition with a polymeric coating or a matrix derived from the polymeric particle, compared to a coating composition or a polymeric matrix composition derived from on a polymeric particle of the same monomer composition polymerized but in a one step emulsion polymerization process.

Disclosed is a polymeric particle with a stepwise or continuous increase of polymerized methacrylic acid units from the center to the surface, obtainable from the disclosed process, for use as coating or binding agent in a pharmaceutical or nutraceutical dosage form.

Disclosed is a polymeric particle, preferably a polymeric particle with an allover monomer composition comprising polymerized units of 10 to 30% by weight methyl methacrylate, 50 to 70% by weight methyl acrylate and 5 to 15% by weight methacrylic acid, wherein the concentration of polymerized units of methacrylic acid by weight at the surface is increased by a factor of 1.2 to 5, preferably 1.5 to 4.5 compared to the allover concentration of methacrylic acid by weight in the polymeric particle. The concentration of polymerized units of methacrylic acid by weight at the surface may be determined by calculation.

The allover concentration of methacrylic acid by weight in the polymeric particle is the amount of methacrylic acid by weight calculated on the total amount of monomers by weight. The allover concentration of methacrylic acid by weight is theoretically equal to the concentration that would be achieved homogeneously over the whole polymeric particle derived from a non-inventive bulk or standard one step emulsion polymerization process.

The amount methacrylic acid by weight at the surface may be calculated in the case of a stepwise polymerization by the amount of methacrylic acid by weight in relation to the other monomers used in the polymeric shell of the polymeric core/shell structure (for instance 19% by weight in example 2).

The amount of methacrylic acid by weight at the surface may be calculated in the case of a gradient polymerization by the relation of the monomers in the monomer charging process (from the center to the surface of the polymeric particle) from the last monomer charge.

(for instance 41% by weight in example 3).

The polymeric particle may be further characterised in that the increasing concentration of polymerized units of methacrylic acid from the center to the surface of the particles results in a lowered active ingredient release pH of an active ingredient containing coated composition or an active ingredient containing polymeric matrix composition with a polymeric coating or a matrix derived from or comprising the polymer from the polymeric particle, compared to a coating composition or a polymeric matrix composition, derived from a polymeric particle or comprising the polymer from a polymeric particle of the same monomer composition polymerized in a one step emulsion polymerization process (The term “derived from” shall be understood in the sense of “made from” or “based on”).

### Aqueous Dispersion

Disclosed is an aqueous dispersion comprising water and the polymeric particles. The aqueous dispersion may comprise 10 to 50, preferably 20 to 40% by weight of the polymeric particles.

### Powder or Granulate

The polymeric particles may be converted from the aqueous dispersion to a dry form, preferably to a powder or a granulate, by spray drying, freeze drying, coagulation spray granulation or extrusion of the aqueous dispersion. The resulting granulate or powder may have a particle size D50 in the range from about 0.01 to 5 mm. Powder may have a particle size D50 in the range from about 0.01 up to less than 0.5 mm. Granulates may have a particle size D50 in the range from about 0.5 mm up to 5 mm. The average particle size of granulates is preferably determined by well known sieving methods. The particle size D50 of powder is preferably determined by laser diffraction.

### Dissolution Behavior/Speed of the Polymeric Particles

The dissolution behavior of polymeric particles from a stepwise and a gradient polymerization process and conventional non-inventive polymer particles with the same allover monomer composition was measured as dissolution speed [mg/min×g dry polymer substance] along an ascending pH gradient (dissolution/pH curve). The comparison of polymeric particles from a batch (standard) emulsion polymerization process (non-inventive) with inventive polymer particles from a stepwise and a gradient polymerization process show that the dissolution/pH curve of the inventive polymer particles is shifted almost parallel to pH values which are about 0.5 to 0.7 pH units lower than those of the dissolution/pH curve of the non-inventive polymer particles.

The polymeric particle may be characterized in that the increasing concentration of polymerized units of methacrylic acid from the center to the surface of the particle results in an accelerated dissolution speed compared to a polymeric particle, polymerized by an emulsion polymerization in one step.

The dissolution speed of polymeric particles as disclosed, preferably for polymeric particles with an allover monomer composition by weight comprising polymerized units of 10 to 30% by weight methyl methacrylate, 50 to 70% by weight methyl acrylate and 5 to 15% by weight methacrylic acid, may be in the range of 10 to 50 mg/min/g polymer at pH 6.5 and/or in the range more than 50 and up to 100 mg/min/g polymer at pH 6.8.

The dissolution speed of polymeric particles from a stepwise polymerization as disclosed, preferably for polymeric particles with an allover monomer composition by weight comprising polymerized units of 10 to 30% by weight methyl methacrylate, 50 to 70% by weight methyl acrylate and 5 to 15% by weight methacrylic acid, may be in the range of 10 to 50, preferably 15 to 30 mg/min/g polymer at pH 6.5 and/or in the range of more than 50 and up to 100, preferably 70 to 95 mg/min/g polymer at p.6.8.

The dissolution speed of polymeric particles from a gradient polymerization as disclosed, preferably for polymeric particles with an allover monomer composition by weight comprising polymerized units of 10 to 30% by weight methyl methacrylate, 50 to 70% by weight methyl acrylate and 5 to 15% by weight methacrylic acid, may be in the range of 20 to 50, preferably 30 to 45 mg/min/g polymer at pH 6.5 and/or in the range of more than 50 and up to 100, preferably 70 to 95 mg/min/g polymer at p.6.8.

The dissolution speed is measured by titration of the methacrylic acid groups in the polymer with NaOH at constant pH-value and at room temperature (20 to 25° C., preferred at 22° C.)

### Dosage Form

Disclosed is a Dosage form, comprising a pharmaceutical or nutraceutical active ingredient and a polymeric coating or a polymeric matrix, wherein the polymeric coating or the polymeric matrix is derived from the polymeric particles as disclosed.

A polymeric coating may be derived, for instance, by spray coating of an aqueous dispersion comprising the polymeric particles onto a core comprising a pharmaceutical or nutraceutical active ingredient.

A polymeric matrix may be derived, for instance, from an aqueous dispersion comprising the polymeric particles or by a spray dried powder from such an aqueous dispersion, by methods such as wet or dry granulation, extrusion granulation or powder binding with the addition a pharmaceutical or nutraceutical active ingredient and optionally further pharmaceutical or nutraceutical excipients, such as antioxidants, brighteners, binding agents, flavouring agents, flow aids, fragrances, glidants, penetration-promoting agents, pigments, plasticizers, polymers, pore-forming agents or stabilizers.

The dosage form may be a coated dosage form comprising a core, comprising an active ingredient, preferably a nutraceutical active ingredient and a polymer coating onto the core, wherein the coating comprises a polymer film derived from the aggregation of the polymeric particles during the film forming process. The dosage form may be for instance in the form of a coated or uncoated pellet, a coated or uncoated tablet, a capsule filled with pellets, a sachet and so on.

The dosage form may be a matrix dosage form comprising an active ingredient, preferably a nutraceutical active ingredient, embedded in a polymeric matrix derived from the aggregation of the polymeric particles during the matrix forming process.

### Active Ingredient Release

A dosage form, preferably a coated dosage form, as disclosed may show an active ingredient release of 10% or more, preferably 30% or more, most preferably 40% or more in a pH range from pH 6.2 to 6.5 preferably in a pH range from 6.2 to 6.4.

A dosage form, preferably a coated dosage form, as disclosed, preferably coated with polymeric particles with an allover monomer composition comprising polymerized units of 10 to 30% by weight methyl methacrylate, 50 to 70% by weight methyl acrylate and 5 to 15% by weight methacrylic acid, may show an active ingredient release of 40 to 100, preferably of 70 to 100% at pH 6.8.

The active ingredient release may be determined according to USP (United States Pharmacopeia) 41, method 2, Paddle 100 rpm.

### Pharmaceutical Active Ingredients

The invention is preferably useful for pharmaceutical active ingredients where the total amount of carboxlic groups in the coating formulation or in the polymeric matrix formation shall be kept low but the active ingredient release is intended to start already in the range of pH 6.0 to 6.5.

Therapeutical and chemical classes of pharmaceutical active ingredients used in the dosage forms as disclosed are for instance analgetics, antibiotics or anti-infectives, antibodies, antiepileptics, antigens from plants, antirheumatics, betablocker, benzimidazole derivatives, beta-blocker, cardiovascular drugs, chemotherapeutics, CNS drugs, digitalis glycosides, gastrointestinal drugs, e.g. proton pump inhibitors, enzymes, hormons, liquid or solid natural extracts, oligonucleotides, peptidhormon proteins, therapeutical bacteria, peptides, proteins (metal)salt f.e. aspartates, chlorides, orthates, urology drugs, vaccines

Further examples of pharmaceutical active ingredients may be: acamprosat, aescin, amylase, acetylsalicylic acid, adrenalin, 5-amino salicylic acid, aureomycin, bacitracin, balsalazine, beta carotene, bicalutamid bisacodyl, bromelain, bromelain, budesonide, calcitonin, carbamacipine, carboplatin, cephalosporins, cetrorelix, clarithromycin, chloromycetin, cimetidine, cisapride, cladribine, clorazepate, cromalyn, 1-deaminocysteine-8-D-arginine-vasopressin, deramciclane, detirelix, dexiansoprazole, diclofenac, didanosine, digitoxin and other digitalis glycosides, dihydrostreptomycin, dimethicone, divalproex, drospirenone, duloxetine, enzymes, erythromycin, esomeprazole, estrogens, etoposide, famotidine, fluorides, garlic oil, glucagon, granulocyte colony stimulating factor (G-CSF), heparin, hydrocortisone, human growth hormon (hGH), ibuprofen, ilaprazole, insulin, Interferon, Interleukin, Intron A, ketoprofen, lansoprazole, leuprolidacetat lipase, lipoic acid, lithium, kinin, memantine, mesalazine, methenamine, methylphenidate, milameline, minerals, minoprazole, naproxen, natamycin, nitrofurantion, novobiocin, olsalazine, omeprazole, orothates, pancreatin, pantoprazole, parathyroidhormone, paroxetine, penicillin, perprazol, pindolol, polymyxin, potassium, pravastatin, prednisone, preglumetacin progabide, pro-somatostatin, protease, quinapril, rabeprazole, ranitidine, ranolazine, reboxetine, rutosid, somatostatin streptomycin, subtilin, sulfasalazine, sulphanilamide, tamsulosin, tenatoprazole, thrypsine, valproic acid, vasopressin, vitamins, zinc, including their salts, derivatives, polymorphs, isomorphs, or any kinds of mixtures or combinations thereof.

### Nutraceutical Active Ingredients

The invention is preferably useful for nutraceutcal active ingredients where the total amount of carboxlic groups of the polymer in a coating formulation or in a polymeric matrix formation shall be kept low but the active ingredient release is intended to start already in the range of pH 6.0 to 6.5.

Nutraceuticals are well known to the skilled person. Nutraceuticals are often defined as extracts of foods claimed to have medical effects on human health. Thus, nutraceutical active ingredients may display pharmaceutical activities as well: Examples for nutraceutical active ingredients may be resveratrol from grape products as an antioxidant, soluble dietary fiber products, such as psyllium seed husk for reducing hypercholesterolemia, broccoli (sulphane) as a cancer preservative, and soy or clover (isoflavonoids) to improve arterial health. Thus it is clear that many substances listed as nutraceuticals may also be used as pharmaceutical active ingredients.

Depending on the territory, the specific application, the local authority legislation and classification, the same substance may be listed as a pharmaceutical or as a nutraceutical active ingredient respectively as a pharmaceutical or a nutraceutical composition or even both. Thus it is evident to a skilled person that there is a broad overlap between the terms pharmaceutical or a nutraceutical active ingredient respectively a pharmaceutical or a nutraceutical composition.

Nutraceuticals or nutraceutical active ingredients are sometimes defined as extracts of foods claimed to have medical effects on human health.

Nutraceuticals or nutraceutical active ingredients may also include probiotics and prebiotics. Probiotics are living microorganisms believed to support human or animal health when consumed, for example certain strains of the genera Lactobacillus or Bifidobacterium. Prebiotics are nutraceuticals or nutraceutical active ingredients that induce or promote the growth or activity of beneficial microorganisms in the human or animal intestine.

The nutraceutical active ingredient may be usually contained in a medical format such as capsule, tablet or powder in a prescribed dose. Examples for nutraceuticals are resveratrol from grape products or pro-anthocyanines from blueberries as antioxidants, soluble dietary fiber products, such as psyllium seed husk for reducing hypercholesterolemia, broccoli (sulphane) as a cancer preservative, and soy or clover (isoflavonoids) to improve arterial health. Other nutraceuticals examples are flavonoids, antioxidants, alpha-linoleic acid from flax seed, beta-carotene from marigold petals or antocyanins from berries. Sometimes the expression neutraceuticals or nutriceuticals are used as synonyms for nutraceuticals.

### EXAMPLES

### Example 1 (Comparative): Standard Emulsion Polymerization of EUDRAGIT® FS 30 D

2.82 g methacrylic acid, 7.46 g methyl methacrylate and 19.29 g methyl acrylate are mixed and continuously charged into 69.8 g water of 75° C., while stirring. The charging is completed after 60 minutes. After that the temperature of 75° C. is kept for additional 60 minutes. The monomers polymerize and form a 30% by weight aqueous dispersion. As excipients 0.07 g Sodium persulfate, 0.08 g 2-ethylhexylthioglycolate, 0.1 g sodium dodecyl sulfate and 0.35 g polysorbate 80 are used.

Result is an aqueous dispersion wherein the monomers are homogeneous distributed in the polymer particles. The content of methacrylic acid is 10% by weight.

### Example 2 (Inventive): Stepwise Emulsion Polymerization of the EUDRAGIT® FS Polymer Type

4.17 g methyl methacrylate and 10.77 g methyl acrylate are mixed and continuously charged into 69.8 g water of 75° C., while stirring. The charging is completed after 30 minutes. The monomers polymerize and form an aqueous dispersion. In a second step 2.82 g methacrylic acid, 3.32 g methyl methacrylate and 8.52 g methyl acrylate are mixed and continuously charged into the dispersion. The second charging is completed after 30 minutes. After that the temperature of 75° C. is kept for additional 60 minutes. The monomers polymerize and finally form a 30% by weight aqueous dispersion. As excipients 0.07 g sodium persulfate, 0.08 g 2-ethylhexylthioglycolate, 0.1 g sodium dodecyl sulfate and 0.35 g Polysorbate 80 are used.

Result is an aqueous dispersion wherein the polymer particles have a core shell structure, with all methacrylic acid in the shell. The shell contains about 19% by weight of methacrylic acid. However, the overall composition is equal to example 1.

### Example 3 (Inventive): Gradient Emulsion Polymerization of the EUDRAGIT® FS Polymer Type

7.46 g methyl methacrylate and 19.29 g methyl acrylate are mixed and continuously charged into 69.8 g water of 75° C., while stirring. During the charge 2.82 g methacrylic acid are continuously charged into the methyl methacrylate and methyl acrylate mixture. The charging is completed after 60 minutes. After that the temperature of 75° C. is kept for additional 60 minutes. The monomers polymerize and form a 30% aqueous dispersion. As excipients 0.07 g sodium persulfate, 0.08 g 2-ethylhexylthioglycolate, 0.1 g sodium dodecyl sulfate and 0.35 g Polysorbate 80 are used.

Result is an aqueous dispersion wherein the monomer composition changes in the polymer particles. Content of methacrylic acid rises from 0.4% (after 2 min) by weight in the center of the particles to approximately 41% at the surface (after 60 min). However, the overall composition is equal to example 1.

TABLE 1
Theoretical development of the composition of the
polymer particles of example 3 during the monomer charging
process from the center to the surface of the polymer.

| Monomer | methyl | methyl | methacrylic | |
|---|---|---|---|---|
| charging time | acrylate | methacrylate | acid | |
| [min] | [%] | [%] | [%] | |
| 2 | 71.9 | 27.7 | 0.4 | Start |
| | | | | (center) |
| 4 | 71.7 | 27.6 | 0.8 | |
| 6 | 71.4 | 27.5 | 1.2 | |
| 8 | 71.1 | 27.3 | 1.6 | |
| 10 | 70.8 | 27.2 | 2.0 | |
| 12 | 70.4 | 27.1 | 2.5 | |
| 14 | 70.1 | 27.0 | 2.9 | |
| 16 | 69.7 | 26.8 | 3.4 | |
| 18 | 69.4 | 26.7 | 3.9 | |
| 20 | 69.0 | 26.5 | 4.5 | |
| 22 | 68.6 | 26.4 | 5.0 | |
| 24 | 68.2 | 26.2 | 5.6 | |
| 26 | 67.8 | 26.1 | 6.2 | |
| 28 | 67.3 | 25.9 | 6.8 | |
| 30 | 66.8 | 25.7 | 7.5 | |
| 32 | 66.3 | 25.5 | 8.2 | |
| 34 | 65.7 | 25.3 | 9.0 | |
| 36 | 65.1 | 25.0 | 9.8 | |
| 38 | 64.5 | 24.8 | 10.7 | |
| 40 | 63.8 | 24.5 | 11.7 | |
| 42 | 63.0 | 24.2 | 12.7 | |
| 44 | 62.2 | 23.9 | 13.9 | |
| 46 | 61.2 | 23.5 | 15.2 | |
| 48 | 60.1 | 23.1 | 16.7 | |
| 50 | 58.9 | 22.6 | 18.5 | |
| 52 | 57.3 | 22.1 | 20.6 | |
| 54 | 55.4 | 21.3 | 23.3 | |
| 56 | 52.6 | 20.2 | 27.1 | |
| 58 | 47.9 | 18.4 | 33.7 | |
| 60 | 42.6 | 16.4 | 41.1 | Final |
| | | | | Produkt |
| | | | | (surface) |

TABLE 2
Dissolution speed of the polymers from examples 1 to 3
in (mg/min/g polymer) at certain pH values
(Method: Titration of methacrylic acid groups in the
polymer with NaOH at constant pH-value)

| pH-value | Example 1 | Example 2 | Example 3 |
|---|---|---|---|
| 5.8 | 0 | 0 | 0 |
| 6 | 1 | 0 | 0 |
| 6.2 | 1 | 0 | 17 |
| 6.5 | 0 | 24 | 44 |
| 6.8 | 3 | 89 | 92 |
| 7 | 22 | 101 | 118 |
| 7.2 | 85 | 120 | |
| 7.5 | 118 | | |

Result: The dissolution speed in inventive example 2 and 3 (stepwise/gradient polymerization) is accelerated compared to the standard EUDRAGIT® FS 30 D product (standard emulsion polymerization) from example 1. The dissolution speed is faster in example 3 (gradient polymerization) compared to example 2 (stepwise polymerization).

### Example 4 (Comparative): Standard Emulsion Polymerization (EUDRAGIT® L 30 D-55

15 g methacrylic acid and 15 g ethyl acrylate are mixed and continuously charged into 69.8 g water of 80° C., while stirring. The charging is completed after 60 minutes. After that the temperature of 80° C. is kept for additional 60 minutes. The monomers polymerize and form a 30% by weight aqueous dispersion. As excipients ammonium persulfate. 2-ethylhexylthioglycolate. sodium dodecyl sulfate and Polysorbate 80 are used.

Result is an aqueous dispersion wherein the monomers are homogeneous distributed in the polymer particles. Content of methacrylic acid is 50% by weight.

### Example 5 (Comparative): Gradient Emulsion Polymerization of the EUDRAGIT® L 30 D-55

15 g ethyl acrylate are continuously charged into 69.8 g water of 80° C., while stirring. During the charge 15 g methacrylic acid are continuously charged into the ethyl acrylate. The charging is completed after 60 minutes. After that the temperature of 80° C. is kept for additional 60 minutes. The monomers polymerize and form a 30% aqueous dispersion. As excipients ammonium persulfate. 2-ethylhexylthioglykolate. sodium dodecyl sulfate and Polysorbate 80 are used.

Result is an aqueous dispersion wherein the monomer composition changes within the polymer particles from the center to the surface. The content of methacrylic acid rises from 0% in the center of the particles to approximately 63% at the surface. However, the overall composition is equal to example 1.

TABLE 3
Dissolution speed of the polymers of comparative examples
4 and 5 in [mg/min/g polymer] at certain pH values
(Method: Titration of the methacrylic acid groups in the
polymer with NaOH at constant pH-value)

| pH-value | Example 4 | Example 5 |
|---|---|---|
| 4 | 2 | 0 |
| 4.5 | 5 | 0 |
| 5 | 6 | 5 |
| 5.5 | 30 | 27 |
| 5.8 | 232 | 95 |
| 6 | 286 | 139 |
| 6.2 | | 187 |
| 6.5 | | 269 |

Result: The dissolution speed in example 5 (gradient polymerization) is not accelerated compared to the standard EUDRAGIT® L 30 D-55 product (standard emulsion polymerization).

### Example 6 (Comparative): Coating of Diprophylline Pellets with Example 1 Polymer Dispersion

100 g of Example 1 polymer dispersion was used to coat 150 g of diprophylline pellets in a Hüttlin Microlab fluidized bed coater. As excipients 15 g talc and 1.5 g triethyl citrate were used.

### Example 7 (Inventive): Coating of Diprophylline Pellets with Example 2 Polymer Dispersion

100 g of Example 2 polymer dispersion was used to coat 150 g of diprophylline pellets in a Hüttlin Microlab fluidized bed coater. As excipients 15 g talc and 1.5 g triethyl citrate were used.

### Example 8 (Inventive): Coating of Diprophylline Pellets with Example 3 Polymer Dispersion

100 g of Example 3 polymer dispersion was used to coat 150 g of diprophylline pellets in a Hüttlin Microlab fluidized bed coater. As excipients 15 g talc and 1.5 g triethyl citrate were used.

TABLE 4
Diprophylline drug release [%] of the
coated pellets of examples 6 to 8
Drug release according to USP 41 method 2,
Paddle 100 rpm, pH 1.0, 6.8 and 7.4

| Time [min] | pH-value | Example 6 | Example 7 | Example 8 |
|---|---|---|---|---|
| 0 | 1.0 | 0 | 0 | 0 |
| 15 | 1.0 | 0.03 | 0 | 0 |
| 30 | 1.0 | 0.06 | 0 | 0 |
| 60 | 1.0 | 0.11 | 0.02 | 0 |
| 90 | 1.0 | 0.16 | 0.03 | 0.01 |
| 120 | 1.0 | 0.23 | 0.04 | 0.03 |
| 140 | 6.8 | 0.29 | 99.35 | 99.69 |
| 150 | 6.8 | 0.34 | 99.56 | 99.7 |
| 165 | 6.8 | 0.47 | 99.57 | 99.72 |
| 180 | 6.8 | 1.21 | 99.64 | 99.72 |
| 210 | 7.4 | 100 | 100.12 | 99.91 |
| 240 | 7.4 | 100 | 100 | 100 |
| 270 | 7.4 | 100 | 100.25 | 99.97 |
| 300 | 7.4 | 100 | 100.06 | 100 |

Result: The drug release of the pellets from the inventive examples 7 and 8 occurs already at pH 6.8 compared to comparative example 6, where the drug release starts at pH 7.4.

1: A process for preparing polymeric particles, comprising polymerized units of methacrylic acid and further monomers, with an overall monomer composition by weight comprising polymerized units of 5 to 25% by weight of methacrylic acid and 75 to 95% by weight of further monomers, wherein the further monomers are selected from the group consisting of C₁- to C₄-alkylesters of methacrylic acid, C₁- to C₄-alkylesters of acrylic acid, and mixtures thereof; the process comprising:
polymerizing units of methacrylic acid and further monomers by a stepwise or gradient emulsion polymerization,
wherein a ratio by weight of polymerized units of methacrylic acid to further monomers is increasing stepwise or in a gradient from the center to the surface of the particles, and
wherein the polymeric particles are obtained in the form of an aqueous dispersion.

2: The process according to claim 1, wherein the polymeric particles are polymeric particles with an overall monomer composition by weight comprising polymerized units of 10 to 30% by weight of methyl methacrylate, 50 to 70% by weight of methyl acrylate and 5 to 15% by weight of methacrylic acid.

3: The process according to claim 1, wherein the stepwise or gradient emulsion polymerization is a stepwise emulsion polymerization comprising at least a first and a second step,
wherein in the first step polymeric core particles are polymerized, wherein a ratio by weight of methacrylic acid to the further monomers is lower compared to the overall monomer composition by weight of methacrylic acid and the further monomers, and
wherein in the second step a polymeric shell is polymerized onto the polymeric core particles wherein a ratio by weight of methacrylic acid to the further monomers is higher compared to the overall monomer composition by weight of methacrylic acid and the further monomers.

4: The process according to claim 1, wherein the stepwise or gradient emulsion polymerization is a stepwise emulsion polymerization with two steps,
wherein in the first step the further monomers are polymerized as polymeric core particles, and
wherein in the second step methacrylic acid is added and polymerized as a polymeric shell onto the polymeric core particles.

5: The process according to claim 1, wherein the stepwise or gradient emulsion polymerization is a gradient emulsion polymerization,
wherein the monomers are polymerized in a continuous process,
wherein the ratio by weight of the methacrylic acid to the further monomers is continuously increased during the polymerization process.

6: The process according to claim 1, wherein the polymeric particles are converted from the aqueous dispersion to a dry form, by spray drying, freeze drying, spray granulation or extrusion of the aqueous dispersion.

7: A polymeric particle, obtainable in the process according to claim 1, comprising polymerized units of 10 to 30% by weight of methyl methacrylate, 50 to 70% by weight of methyl acrylate and 5 to 15% by weight of methacrylic acid, with a stepwise or continuous increase of the polymerized methacrylic acid units from the center to the surface.

8: The polymeric particle according to claim 7, wherein an average particle size is in the range from about 50 to 500 nm.

9: The polymeric particle according to claim 7, wherein an increasing concentration of polymerized units of methacrylic acid from the center to the surface of the particle results in an accelerated dissolution speed compared to a polymeric particle polymerized by an emulsion polymerization in one step.

10: The polymeric particle according to claim 7, wherein an increasing concentration of polymerized units of methacrylic acid from the center to the surface of the particle results in a lowered active ingredient release pH of an active ingredient containing coated composition, an active ingredient containing polymeric matrix composition with a polymeric coating, or a matrix derived from the polymeric particle, compared to a coating composition or a polymeric matrix composition derived from a polymeric particle of the same monomer composition polymerized in a one step emulsion polymerization process.

11: The polymeric particle according to claim 7, wherein a concentration of polymerized units of methacrylic acid by weight at the surface is increased by a factor of 1.2 to 5 compared to a concentration of methacrylic acid content by weight in the allover monomer composition of the polymeric particle.

12: A method, comprising:
using the polymeric particle according to claim 7 as a coating or binding agent in a pharmaceutical or nutraceutical dosage form.

13: An aqueous dispersion, comprising water and the polymeric particle according to claim 7.

14: The aqueous dispersion according to claim 13, comprising 10 to 50% by weight of the polymeric particle.

15: A dosage form, comprising a pharmaceutical or nutraceutical active ingredient and a polymeric coating or a polymeric matrix,
wherein the polymeric coating or the polymeric matrix is derived from the polymeric particle according to claim 7.

16: The process according to claim 4, wherein the further monomers are methyl methacrylate and methyl acrylate.

17: The process according to claim 6, wherein the dry form is a powder or a granulate.

18: The process according to claim 8, wherein the average particle size is in the range from about 80 to 300 nm.

19: The polymeric particle according to claim 11, wherein the concentration of polymerized units of methacrylic acid by weight at the surface is increased by a factor of 1.5 to 4.5 compared to the concentration of methacrylic acid content by weight in the allover monomer composition of the polymeric particle.
